(19) United States
(12) Patent Application Publication
(10) Pub. No.: US 2019/0030107 A1
(43) Pub. Date: Jan. 31, 2019

(54) COMPOSITIONS FOR PERIODONTAL ADJUNTIVE THERAPY

(71) Applicants: Ragan L. Faler, Fayetteville, GA (US); Lowery A. Faler, Hattiesburg, MS (US)

(72) Inventors: Ragan L. Faler, Fayetteville, GA (US); Lowery A. Faler, Hattiesburg, MS (US)

(21) Appl. No.: 15/664,356

(22) Filed: Jul. 31, 2017

(51) Int. Cl. A61K 36/534; A61K 33/00; A61K 33/10; A61K 31/05; A61K 36/54; A61K 47/10; A61K 31/4164; A61K 36/61; A61K 9/00; A61K 6/00

(52) U.S. Cl. CPC A61K 36/534; A61K 33/00; A61K 33/10; A61K 31/05; A61K 6/0035; A61K 47/10; A61K 31/4164; A61K 36/61; A61K 9/0053; A61K 36/54

(57) ABSTRACT

The scaling, root planning, and curettage procedure, even without additional treatment, benefits the patient by providing a reduction in pocket depth. The present disclosure provides high pH periodontal adjuvant compositions that, in conjunction with scaling and root planning, can reduce or eliminate a population of bacteria from a site of periodontal treatment. The compositions are applied as a paste and removed by lavage, thereby reducing or removing an active periodontal infection and allowing the periodontium to return to a state of health.

### TECHNICAL FIELD

The present disclosure is generally related to compositions and methods for reducing an oral microbial population at a site of periodontal therapy.

### BACKGROUND

The most commonly encountered diseases of the periodontium are gingivitis and periodontitis. The roles of microbial etiology and host response are well recognized in the course of progression of these diseases. Periodontal treatments range from scaling and root planning (SRP) to surgical interventions. Adjunctive therapy refers to an intervention that is additional to the primary treatment and is performed to enhance the results of the primary treatment. Adjunctives are administered to attain the most excellent results possible by reducing the inflammatory burden on the treated region. These effects are produced by administering agents that modulate the immune responses, fight inflammatory changes, and prevent infections by obliterating bacterial load. A number of periodontal adjunct modalities are being used currently while several improved versions are being developed. Currently, these adjunct therapies are broadly categorized as irrigation, antimicrobial therapy, host modulation, lasers, and local adjuncts (Bader, H. (2011) Adjunctive Periodontal Therapy: Rev. Curr. Tech. 1st ed: Chicago: Dentistry Today).

The most popular adjuncts are those locally administered. Local agents are applied into the pockets of the affected tooth/teeth. These agents are antimicrobial substances supplied in various forms. Currently, a number of these agents are available in the market. As these therapies have been implemented, results have still varied with the efficacy of each of the adjuncts as previously discussed. As one reviews the dental product market, the sheer number of available and previously available products proves that a definitive treatment and cure of periodontal disease has alluded clinicians at times.

The cornerstone of controlling periodontitis is thorough scaling and planing of root surfaces in order to reduce bacterial loads that cause inflammatory response. The effectiveness of scaling and root planing is due in part to operator skill level, patient host response, and accessibility or visibility due to tooth morphology or depth of the pocket. A number of studies have documented the clinical efficacy of SRP in combination with systemically or locally delivered antimicrobial agents (Feres et al., (1999) J. Clin. Periodontol. 26: 775-783; Flemmig et al., (1998) J. Clin. Periodontol. 25: 380-387; Soder et al., (1990) J. Periodontol. 61: 281-28). Practitioners have traditionally chosen the antimicrobial agents based more on personal experience than on evidence from microbiological studies and rigorous clinical trials. Also, success is generally measured in observed pocket depth reduction vs. tooth retention or reduction of pathogenic bacteria.

### SUMMARY

Briefly, one aspect of the disclosure encompasses embodiments of a periodontal adjunct composition comprising at least one alkali metal hydroxide, a metal salt, a phenolic compound, and an essential oil.

In some embodiments of this aspect of the disclosure, the periodontal adjunct composition can further comprises an excipient.

In some embodiments of this aspect of the disclosure, the composition can further comprise an H₂ receptor antagonist.

In some embodiments of this aspect of the disclosure, the H₂ receptor antagonist can be selected from the group consisting of: cimetidine, nizatidine, famotidine, and ranitidine.

In some embodiments of this aspect of the disclosure, the at least one alkali metal hydroxide can be selected from the group consisting of potassium hydroxide, sodium hydroxide, calcium hydroxide, lithium hydroxide, and rubidium hydroxide.

In some embodiments of this aspect of the disclosure, the phenolic compound can be selected from the group consisting of thymol, carvacrol, cresol, estradiol, eugenol, gallic acid, guaiacol, methyl salicylate, raspberry ketone, salicylic acid, sesamol, tumeric, phenol, bisphenol A, BHT, 4-nonylphenol, orthophenyl phenol, phenolphthalein, and xylenol.

In some embodiments of this aspect of the disclosure, the essential oil can be selected from the group consisting of cinnamon oil, clove oil, eucalyptus oil, peppermint oil, oregano oil, lavender oil, tea tree oil, bergamont oil, lemongrass oil, thyme oil, basil oil, and rosemary oil.

In some embodiments of this aspect of the disclosure, the excipient can be glycerin.

In some embodiments of this aspect of the disclosure, the periodontal adjunct composition is a paste.

In some embodiments of this aspect of the disclosure, the periodontal adjunct composition of claim 1, the at least one alkali metal hydroxide can be between about 85% and about 95% by weight; the metal salt can be between about 1% and about 5% by weight; the phenolic compound can be between about 1% and about 5% by weight; and the essential oil can be between about 1% and about 5% by weight.

In some embodiments of this aspect of the disclosure, the H₂ receptor antagonist can be between about 0.1% and about 1% by weight.

In some embodiments of this aspect of the disclosure, the at least one alkali metal hydroxide can be between about 50% and about 95% by weight; the metal salt can be between about 0.5% and about 5% by weight; the phenolic compound can be between about 0.5% and about 5% by weight; the essential oil is between about 0.5% and about 5% by weight; the glycerin can be between about 5% and about 50% by weight, and the composition can further comprise between about 0.1% and about 1% by weight of an H₂ receptor antagonist.

In some embodiments of this aspect of the disclosure, the at least one alkali metal hydroxide can be about 90% by weight; the metal salt can be about 3% by weight; the phenolic compound can be about 3% by weight; and the essential oil can be about 3% by weight.

In some embodiments of this aspect of the disclosure, the at least one alkali metal hydroxide can be about 60% by weight; the metal salt can be about 2% by weight; the phenolic compound can be about 2% by weight; the essential oil can be about 2% by weight; the H₂ receptor antagonist can be between about 0.1% and about 1% by weight; and the composition can further comprise about 40% by weight of an excipient.

In some embodiments of this aspect of the disclosure, the at least one alkali metal hydroxide can be potassium hydroxide, the metal salt can be magnesium carbonate, the phenolic compound can be thymol, and the essential oil can be cinnamon oil, clove oil, or peppermint oil.

In some embodiments of this aspect of the disclosure, the at least one alkali metal hydroxide can be potassium hydroxide, the phenolic compound can be thymol, the essential oil can be one of cinnamon oil, clove oil, and peppermint oil, and the excipient can be glycerin.

In some embodiments of this aspect of the disclosure, the periodontal adjunct composition can consist of about 60% by weight of potassium hydroxide, about 2% by weight of magnesium carbonate, about 2% by weight of thymol, about 2% by weight of one of cinnamon oil, clove oil, or peppermint oil, about 0.33% by weight of an H₂ receptor antagonist, and about 33% by weight of glycerin.

Another aspect of the disclosure encompasses embodiments of a method of reducing an oral microbial population at a site of periodontal treatment, comprising the steps of: (a) applying to the surface of a site of periodontal treatment of a patient an amount of a periodontal adjunct composition comprising: between about 50% and about 70% by weight of at least one alkali metal hydroxide; between about 0.5% and about 5% by weight of a metal salt; between about 0.5% and about 5% by weight of a phenolic compound; between about 0.5% and about 5% by weight of an essential oil; and between about 5% and about 40% by weight of an excipient, wherein the periodontal adjunct composition is a paste; and (b) rinsing the periodontal adjunct composition from the site of application with a physiologically acceptable liquid.

In some embodiments of this aspect of the disclosure, the periodontal adjunct composition can further comprise between about 0.1% and about 1% by weight of an H₂ receptor antagonist;

In some embodiments of this aspect of the disclosure, the method can further comprise the step of obtaining the periodontal adjunct composition by admixing the at least one alkali metal hydroxide, the metal salt, the phenolic compound, the essential oil, and the excipient to form a paste.

In some embodiments of this aspect of the disclosure, the at least one alkali metal hydroxide can be selected from the group consisting of potassium hydroxide, sodium hydroxide, calcium hydroxide, lithium hydroxide, and rubidium hydroxide; the phenolic compound can be selected from the group consisting of thymol, carvacrol, cresol, estradiol, eugenol, gallic acid, guaiacol, methyl salicylate, raspberry ketone, salicylic acid, sesamol, tumeric, phenol, bisphenol A, BHT, 4-nonylphenol, orthophenyl phenol, phenolphthalein, and xylenol; and the essential oil can be selected from the group consisting of cinnamon oil, clove oil, eucalyptus oil, peppermint oil, oregano oil, lavender oil, tea tree oil, bergamont oil, lemongrass oil, thyme oil, basil oil, and rosemary oil.

In some embodiments of this aspect of the disclosure, the at least one alkali metal hydroxide can be potassium hydroxide, the metal salt can be magnesium carbonate the phenolic compound can be thymol, the essential oil can be one of cinnamon oil, clove oil, or peppermint oil, and the excipient can be glycerin.

In some embodiments of this aspect of the disclosure, the periodontal adjunct composition can consist of about 60% by weight of potassium hydroxide, about 2% by weight of magnesium carbonate, about 2% by weight of thymol, about 2% by weight of cinnamon oil, clove oil, or peppermint oil, and about 33% by weight of glycerin.

In some embodiments of this aspect of the disclosure, the periodontal adjunct composition can consist of about 60% by weight of potassium hydroxide, about 2% by weight of magnesium carbonate, about 2% by weight of thymol, about 2% by weight of cinnamon oil, clove oil, or peppermint oil, about 0.33% of an H₂ receptor antagonist, and about 50% by weight of glycerin.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1A is an analysis of the microbial population of a site of periodontal treatment of Patient 1 before curretage treatment with the composition of the disclosure. Microbial species specifically tested for were: Pg, Porphyromonas gingivalis; Tf, Tannerella forsythia; Td, Treponema denticola; En, Eubacterium nodatum; Cr, Campylobacter rectus; Pi, Prevotella intermedia; Fn, Fusobacterium nucleatum/periodonticum; Pm, Peptostreptococcus (Micromonas) micros; Cs, Capnocytophaga spp (gingavalis, ochracea, sputigena). DL is the detection level of the assay used. The heavy horizontal bar is the threshold level above which the patient is at increased risk of periodontal detachment.

FIG. 1B is an analysis of the microbial population of a site of periodontal treatment of Patient 1 eleven days after curretage treatment with the composition of the disclosure.

FIG. 2A is an analysis of the microbial population of a site of periodontal treatment of Patient 1 before curretage treatment with the composition of the disclosure.

FIG. 2B is an analysis of the microbial population of a site of periodontal treatment of Patient 1 thirteen days after curretage treatment with the composition of the disclosure.

### DETAILED DESCRIPTION

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

Before the embodiments of the present disclosure are described in detail, it is to be understood that, unless otherwise indicated, the present disclosure is not limited to particular materials, reagents, reaction materials, manufacturing processes, dimensions, frequency ranges, applications, or the like, as such can vary. It is also to be understood that the terminology used herein is for purposes of describing particular embodiments only and is not intended to be limiting. It is also possible in the present disclosure that steps can be executed in different sequence, where this is logically possible. It is also possible that the embodiments of the present disclosure can be applied to additional embodiments involving measurements beyond the examples described herein, which are not intended to be limiting. It is furthermore possible that the embodiments of the present disclosure can be combined or integrated with other measurement techniques beyond the examples described herein, which are not intended to be limiting.

It should be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. Further, documents or references cited in this text, in a Reference List before the claims, or in the text itself; and each of these documents or references (“herein cited references”), as well as each document or reference cited in each of the herein-cited references (including any manufacturer's specifications, instructions, etc.) are hereby expressly incorporated herein by reference.

Prior to describing the various embodiments, the following definitions are provided and should be used unless otherwise indicated.

### Definitions

The term “reducing an oral microbial population” shall be understood to include reduction to a certain decreased level as well as reduction to zero, i.e. complete elimination of viable microorganisms.

The “site of periodontal treatment” as used herein refers to particularly the individual's teeth and periodontal regions in the mouth. Especially the individual's teeth down to the gingival margin and/or the periodontal pockets of the individual's mouth shall be treated as well as root canals in endodontic treatment.

### Discussion

The scaling, root planning, and curettage procedure, even without additional treatment, benefits the patient by providing a reduction in pocket depth. However, there is a continuing search to find methods of increasing the level of benefit and prolonging the period of effectiveness. The present disclosure provides compositions that, in conjunction with scaling and root planning, are advantageous to reduce a population of bacteria from a periodontal pocket at a site of periodontal treatment and thus removing the active periodontal infection and allowing the periodontium to return to a state of health.

Generally and preferably, the compositions of the application are in the form of a paste, an aliquot (between about 50 μl to about 200 μl in volume, larger if the volume of the treated site is greater than that of one tooth location) of which can be placed on a dental curette and introduced into a treated periodontal pocket during the planing of the tooth root. Typically, it has been found that a 5-10 second curettage with the present compositions followed by saline irrigation and removal of the paste along with any debris or deposits results in a prolonged decrease in the level of microbial colonization or infection of the treated site and increases the likelihood and degree of success in preventing an infection after periodontal implanting. The irrigation allows the applied alkaline paste to be diluted so that no adverse action of the high pH upon sensitive gingival tissues can occur.

Accordingly, the present disclosure provides embodiments of a composition for use in the prevention and amelioration of a microbial population that may adversely affect the success of a periodontal treatment, in particular the outcome of inserting a periodontal implant in the jaw of a patient. The periodontal adjunct compositions are compounded to include at least one, and preferably a plurality, of alkaline agents, so as to have a pH prior to introduction into the oral environment of at least 9.0-11.0.

The compositions of the disclosure can be advantageously compounded without an excipient and then may be stored for eventual preparation as a paste prior to application to a patient. It is contemplated, however, that the composition may also be admixed as a paste with an excipient and may then be conveniently stably stored for a period of days for application to one or more patients.

It is further contemplated that the compositions of the disclosure may also include additives such as plasticizers, colorants, preservants, and protectants to enhance aesthetic, and most significantly, mechanical features (i.e. softness and flexibility). The type and amount of plasticizer, additive, colorant, preservant, and/or protectant to be used can be readily optimized to achieve a desired effect. Also, surfactants and drying agents may be added as well to the composition to achieve a desired effect. The present pharmaceutical compositions may also contain taste modifiers to enhance or add to the taste benefits arising from the use of essential oils such as peppermint oil, clove oil, and the like, coloring agents, and moisture retaining agent. Examples of taste modifiers include non-reducing sugars, such as xylitol and malitol. Examples of moisture retaining agents include celluloses, cellulose derivatives (for example, methylcellulose), starches, starch derivatives, vegetable gums, non-hygroscopic mono- and di-oligosaccharides, polyethylene glycol, and silicon dioxide.

The methods of the disclosure for treating periodontal disease can optionally further comprise administering to a patient at least one step of sub-gingival scaling, root planning, or curettage. These procedures involve manually removing calculus, plaque and other deposits, smoothing the root surface to rid of necrotic tooth substances, and curetting the inner surface of the gingival wall of the periodontal pockets to separate away the diseased soft tissue.

The compositions of the disclosure preferably include potassium hydroxide as a strongly basic agent that is capable of achieving the desired pH for the composition and retaining a high pH upon introduction into the oral environment, in particular in the moist environment of the periodontal site. Accordingly, preferred excipients for forming the paste compositions are water miscible to allow the potassium hydroxide to ionize to generate the high pH. In addition to potassium hydroxide, the composition preferably includes additional agents such as aluminum hydroxide, calcium carbonate and magnesium carbonate that, while contributing to the maintenance of the high pH, can function as fillers to provide the paste consistency of the applied composition.

The preferred alkali for use in the compositions of the disclosure is potassium hydroxide (KOH). However, other alkaline metal hydroxides can be substituted or included in the compositions of the disclosure including, but not limited to, sodium hydroxide (NaOH), calcium hydroxide (Ca(OH)₂), lithium hydroxide (LiOH), rubidium hydroxide (RbOH), and cesium hydroxide (CsOH). Due to the pH above 7, a strong base of a metal hydroxide can also be used as a substitute or included with such as potassium hydroxide. Metal hydroxide bases suitable for inclusion in the compositions of the disclosure include, but are not limited to, aluminum hydroxide, beryllium hydroxide, cobalt(II) hydroxide, copper(II) hydroxide, curium hydroxide, gold(III) hydroxide, iron(II) hydroxide, mercury(II) hydroxide, nickel(II) hydroxide, tin(II) hydroxide, zinc hydroxide, and zirconium(IV) hydroxide. Most advantageously, the metal hydroxide must provide a localized elevation of the pH to at least 9.0 before lavage from the mouth of the patient.

In addition to the alkaline metal hydroxides and carbonates, it has been found advantageous to include in the compositions at least one phenolic compound that can reduce both inflammation induced by a microbial infection and/or irritation of the sensitive gingival tissue from the high pH of the compositions of the disclosure. Most useful has been the inclusion of thymol, but any phenolic compound may be used that is pharmaceutically acceptable in the amounts that may be applied to a periodontal site by a dental curette and that are known in the art to reduce inflammation.

Accordingly, the compositions of the present disclosure further comprise at least one anti-inflammatory phenolic compound such as, but not limited to, the naturally occurring phenolics thymol (a naturally occurring phenolic compound that has anti-inflammatory properties that aid in soft tissue healing and reduction of inflammatory mediators in periodontitis), carvacrol, cresol, estradiol, eugenol, gallic acid, guaiacol, methyl salicylate, raspberry ketone, salicylic acid, sesamol, and tumeric. Synthetic phenols suitable for use in the compositions of the disclosure include, but are not limited to, phenol, bisphenol A, BHT, 4-nonylphenol, orthophenyl phenol, phenolphthalein, and xylenol.

The compositions of the present disclosure further comprise at least one antibacterial essential oil such as, but not limited to, cinnamon oil, clove oil, eucalyptus oil, peppermint oil, oregano oil, lavender oil, tea tree oil, bergamont oil, lemongrass oil, thyme oil, basil oil, and rosemary oil.

Further included in the compositions of the present disclosure is a metal salt that can serve to enhance the antimicrobial effect when applied to a patient. Salts of silver and copper may reduce the microbial load of a periodontal site. The metal salt such as a calcium or magnesium carbonate can act as a filler, a stabilizer, or assist in the maintenance of the high pH. Accordingly, any metal salt known in the art that is non-toxic to a human or animal patient may be incorporated in the compositions.

The compositions of the disclosure may further include a therapeutic agent such as, but not limited to, an H₂ receptor antagonist that may be incorporated to induce the formation of bone repair and tissue regeneration, thereby improving the outcome of a periodontal implant. Such antagonists, besides their more typical uses as antihistamines, and in particular as inhibitors of acid production by parietal cells, have shown evidence of playing a role in bone metabolism via the H₂ receptor, stimulating bone resorption. Studies have shown that a histamine H₂-receptor antagonist interferes with the initiation and progression of induced periodontal disease in rat molars and that cimetidine can exert a beneficial effect on periodontal disease in rats, and reducing alveolar bone resorption. Suitable H₂ receptor antagonists include, but are not limited to, cimetidine, nizatidine, famotidine, and ranitidine. If included in the compositions of the disclosure, since the pH of the composition is high (greater than 8, and most preferably at least 10), which may be deleterious to the stability of an added therapeutic agent, it is preferred that the agent be contacted with the alkali just before application to a periodontal surface. Accordingly, stock preparations of the compositions without the therapeutic agent may be prepared and mixed with the therapeutic agent and/or the excipient immediately prior to use.

Accordingly, one aspect of the disclosure encompasses embodiments of a periodontal adjunct composition comprising at least one alkali metal hydroxide, a metal salt, a phenolic compound, and an essential oil.

In some embodiments of this aspect of the disclosure, the periodontal adjunct composition can further comprises an excipient.

In some embodiments of this aspect of the disclosure, the composition can further comprise an H₂ receptor antagonist.

In some embodiments of this aspect of the disclosure, the H₂ receptor antagonist can be selected from the group consisting of: cimetidine, nizatidine, famotidine, and ranitidine.

In some embodiments of this aspect of the disclosure, the at least one alkali metal hydroxide can be selected from the group consisting of potassium hydroxide, sodium hydroxide, calcium hydroxide, lithium hydroxide, and rubidium hydroxide.

In some embodiments of this aspect of the disclosure, the phenolic compound can be selected from the group consisting of thymol, carvacrol, cresol, estradiol, eugenol, gallic acid, guaiacol, methyl salicylate, raspberry ketone, salicylic acid, sesamol, tumeric, phenol, bisphenol A, BHT, 4-nonylphenol, orthophenyl phenol, phenolphthalein, and xylenol.

In some embodiments of this aspect of the disclosure, the essential oil can be selected from the group consisting of cinnamon oil, clove oil, eucalyptus oil, peppermint oil, oregano oil, lavender oil, tea tree oil, bergamont oil, lemongrass oil, thyme oil, basil oil, and rosemary oil.

In some embodiments of this aspect of the disclosure, the excipient can be glycerin.

In some embodiments of this aspect of the disclosure, the periodontal adjunct composition is a paste.

In some embodiments of this aspect of the disclosure, the periodontal adjunct composition of claim 1, the at least one alkali metal hydroxide can be between about 85% and about 95% by weight; the metal salt can be between about 1% and about 5% by weight; the phenolic compound can be between about 1% and about 5% by weight; and the essential oil can be between about 1% and about 5% by weight.

In some embodiments of this aspect of the disclosure, the H₂ receptor antagonist can be between about 0.1% and about 1% by weight.

In some embodiments of this aspect of the disclosure, the at least one alkali metal hydroxide can be between about 50% and about 95% by weight; the metal salt can be between about 0.5% and about 5% by weight; the phenolic compound can be between about 0.5% and about 5% by weight; the essential oil is between about 0.5% and about 5% by weight; the glycerin can be between about 5% and about 50% by weight, and the composition can further comprise between about 0.1% and about 1% by weight of an H₂ receptor antagonist.

In some embodiments of this aspect of the disclosure, the at least one alkali metal hydroxide can be about 90% by weight; the metal salt can be about 3% by weight; the phenolic compound can be about 3% by weight; and the essential oil can be about 3% by weight.

In some embodiments of this aspect of the disclosure, the at least one alkali metal hydroxide can be about 60% by weight; the metal salt can be about 2% by weight; the phenolic compound can be about 2% by weight; the essential oil can be about 2% by weight; the H₂ receptor antagonist can be between about 0.1% and about 1% by weight; and the composition can further comprise about 40% by weight of an excipient.

In some embodiments of this aspect of the disclosure, the at least one alkali metal hydroxide can be potassium hydroxide, the metal salt can be magnesium carbonate, the phenolic compound can be thymol, and the essential oil can be cinnamon oil, clove oil, or peppermint oil.

In some embodiments of this aspect of the disclosure, the at least one alkali metal hydroxide can be potassium hydroxide, the phenolic compound can be thymol, the essential oil can be one of cinnamon oil, clove oil, and peppermint oil, and the excipient can be glycerin.

In some embodiments of this aspect of the disclosure, the periodontal adjunct composition can consist of about 60% by weight of potassium hydroxide, about 2% by weight of magnesium carbonate, about 2% by weight of thymol, about 2% by weight of one of cinnamon oil, clove oil, or peppermint oil, about 0.33% by weight of an H₂ receptor antagonist, and about 33% by weight of glycerin.

Another aspect of the disclosure encompasses embodiments of a method of reducing an oral microbial population at a site of periodontal treatment, comprising the steps of: (a) applying to the surface of a site of periodontal treatment of a patient an amount of a periodontal adjunct composition comprising: between about 50% and about 70% by weight of at least one alkali metal hydroxide; between about 0.5% and about 5% by weight of a metal salt; between about 0.5% and about 5% by weight of a phenolic compound; between about 0.5% and about 5% by weight of an essential oil; and between about 5% and about 40% by weight of an excipient, wherein the periodontal adjunct composition is a paste; and (b) rinsing the periodontal adjunct composition from the site of application with a physiologically acceptable liquid.

In some embodiments of this aspect of the disclosure, the periodontal adjunct composition can further comprise between about 0.1% and about 1% by weight of an H₂ receptor antagonist;

In some embodiments of this aspect of the disclosure, the method can further comprise the step of obtaining the periodontal adjunct composition by admixing the at least one alkali metal hydroxide, the metal salt, the phenolic compound, the essential oil, and the excipient to form a paste.

In some embodiments of this aspect of the disclosure, the at least one alkali metal hydroxide can be selected from the group consisting of potassium hydroxide, sodium hydroxide, calcium hydroxide, lithium hydroxide, and rubidium hydroxide; the phenolic compound can be selected from the group consisting of thymol, carvacrol, cresol, estradiol, eugenol, gallic acid, guaiacol, methyl salicylate, raspberry ketone, salicylic acid, sesamol, tumeric, phenol, bisphenol A, BHT, 4-nonylphenol, orthophenyl phenol, phenolphthalein, and xylenol; and the essential oil can be selected from the group consisting of cinnamon oil, clove oil, eucalyptus oil, peppermint oil, oregano oil, lavender oil, tea tree oil, bergamont oil, lemongrass oil, thyme oil, basil oil, and rosemary oil.

In some embodiments of this aspect of the disclosure, the at least one alkali metal hydroxide can be potassium hydroxide, the metal salt can be magnesium carbonate the phenolic compound can be thymol, the essential oil can be one of cinnamon oil, clove oil, or peppermint oil, and the excipient can be glycerin.

In some embodiments of this aspect of the disclosure, the periodontal adjunct composition can consist of about 60% by weight of potassium hydroxide, about 2% by weight of magnesium carbonate, about 2% by weight of thymol, about 2% by weight of cinnamon oil, clove oil, or peppermint oil, and about 33% by weight of glycerin.

In some embodiments of this aspect of the disclosure, the periodontal adjunct composition can consist of about 60% by weight of potassium hydroxide, about 2% by weight of magnesium carbonate, about 2% by weight of thymol, about 2% by weight of cinnamon oil, clove oil, or peppermint oil, about 0.33% of an H₂ receptor antagonist, and about 50% by weight of glycerin.

It should be emphasized that the embodiments of the present disclosure, particularly any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and protected by the following claims.

The specific examples below are to be construed as merely illustrative and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified.

### EXAMPLES

The following examples are presented by way of illustration, not of limitation.

### Example 1

TABLE 1

| Ingredient | Amount |
|---|---|
| Potassium hydroxide | 18.0 g |
| Magnesium carbonate | 0.60 g |
| Thymol | 0.60 g |
| Cinnamon oil | 0.60 g |
| Glycerin | Final weight 30 g |

### Example 2

TABLE 2

| Ingredient | Amount |
|---|---|
| Potassium hydroxide | 18.0 g |
| Magnesium carbonate | 0.60 g |
| Thymol | 0.60 g |
| Cinnamon oil | 0.60 g |
| Cimetidine | 100 mg per 30 g of formula weight |
| Glycerin | Final weight 30 g |

The potassium hydroxide is ground to a fine powder and sifted before weighing. Thymol is also reduced to a powder before blending with the cinnamon oil and magnesium carbonate. The potassium hydroxide is then added.

Glycerin is then added in small portions with mixing after each addition to result in a smooth paste. An advantageous final useful consistency should be that resembling facial cold cream and which will not escape from the dental site of application without lavage. The amounts shown in Table 2 are sufficient to produce about 30 g of the final paste composition.

### Example 3

TABLE 3

| Ingredient | Amount |
|---|---|
| Potassium hydroxide | 18.0 g |
| Magnesium carbonate | 0.60 g |
| Thymol | 0.60 g |
| Peppermint oil | 0.60 g |
| Cimetidine | 100 mg per 30 g of formula weight |
| Glycerin | Final weight 30 g |

### Example 4

TABLE 4

| Ingredient | Amount |
|---|---|
| Potassium hydroxide | 18.0 g |
| Magnesium carbonate | 0.60 g |
| Thymol | 0.60 g |
| Clove oil | 0.60 g |
| Cimetidine | 100 mg per 30 g of formula weight |
| Glycerin | Final weight 30 g |

### Example 5

TABLE 5

| Ingredient | Amount |
|---|---|
| Sodium hydroxide | 18.0 g |
| Magnesium carbonate | 0.60 g |
| Thymol | 0.60 g |
| Peppermint oil | 0.60 g |
| Cimetidine | 100 mg per 30 g of formula weight |
| Glycerin | Final weight 30 g |

### Example 6

The bacterial loadings of teeth of several patients, before and after treatment with a composition of the disclosure, were determined using a PCR-based procedure, providing both species and species load (MYPERIOPATH.®, OralDNA Labs, Access Genetics LLC, Eden Prarie, Minn.).

Briefly, Genomic DNA was extracted from a submitted sample and tested for 10 species-specific bacteria and 1 genus of bacteria known to cause periodontal disease. The bacteria were assayed by real-time quantitative polymerase chain reaction (qPCR). Bacterial loads were reported in log copies per mL of sample (e.g. 1×10³=1000 bacteria copies per mL of collection). (OraIDNA Labs, Access genetics, LLC, Eden Prarie, Minn. and modified from Periodontology 2000 (2006) 42: 180-218.)

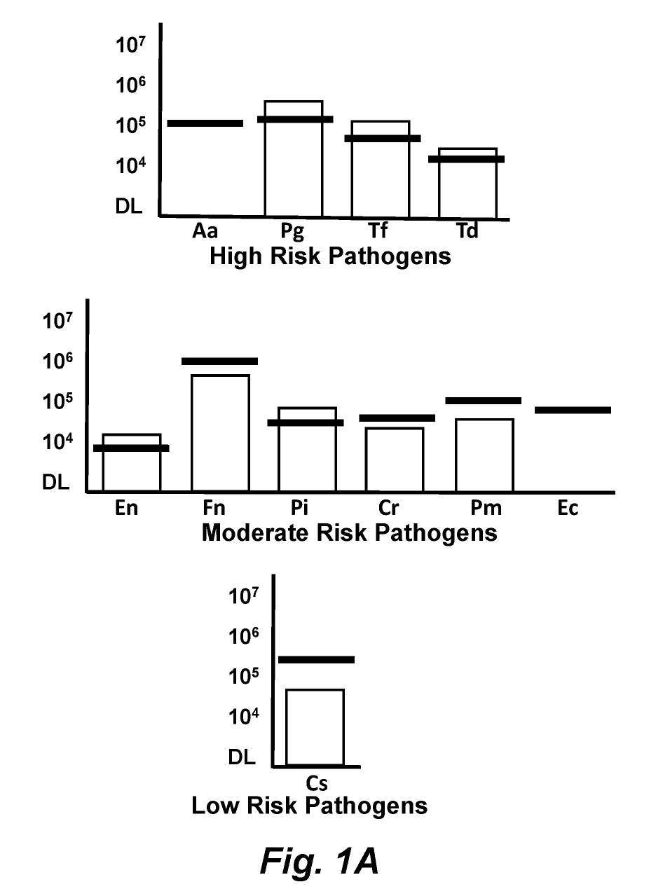

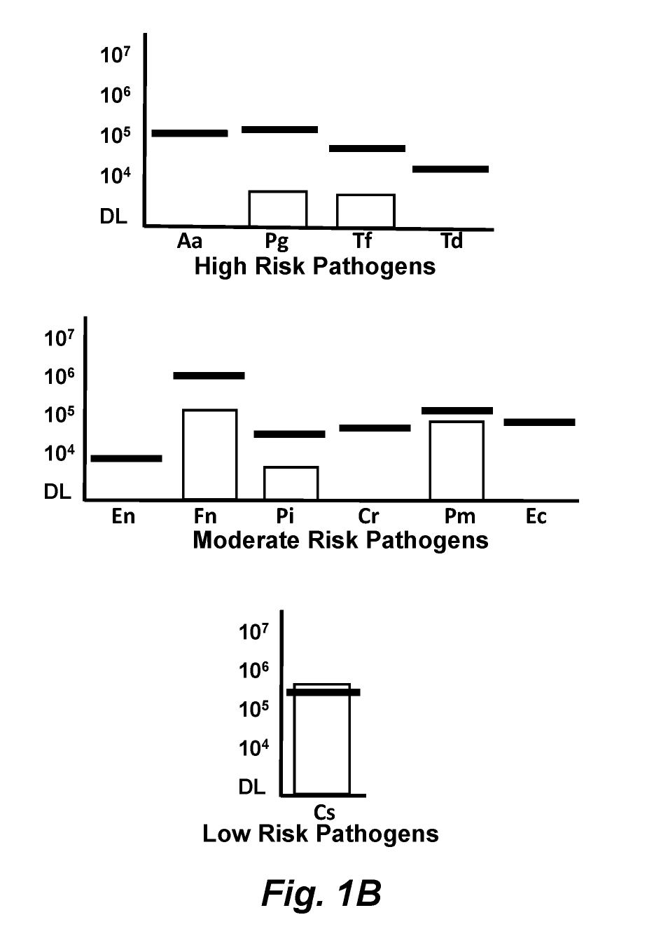

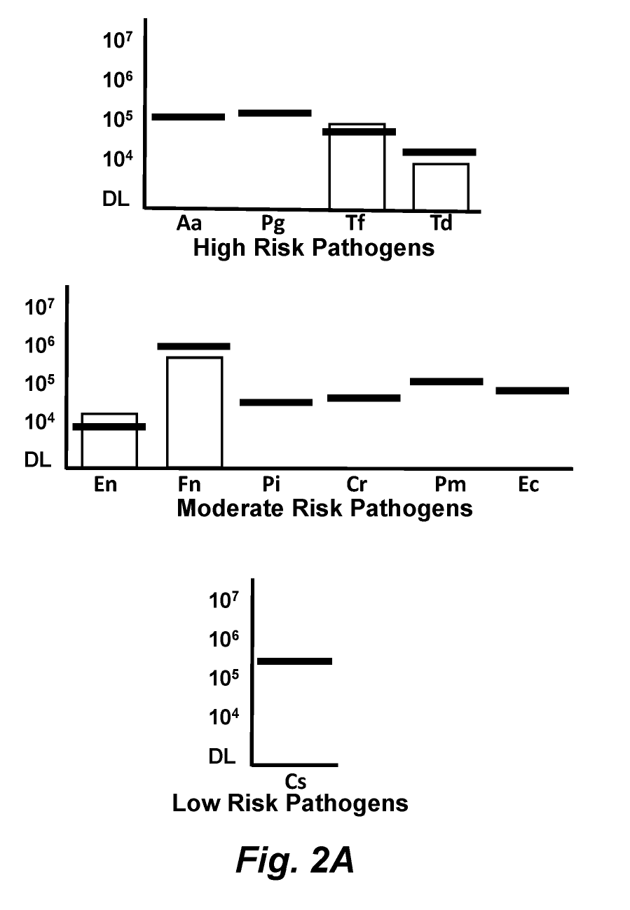

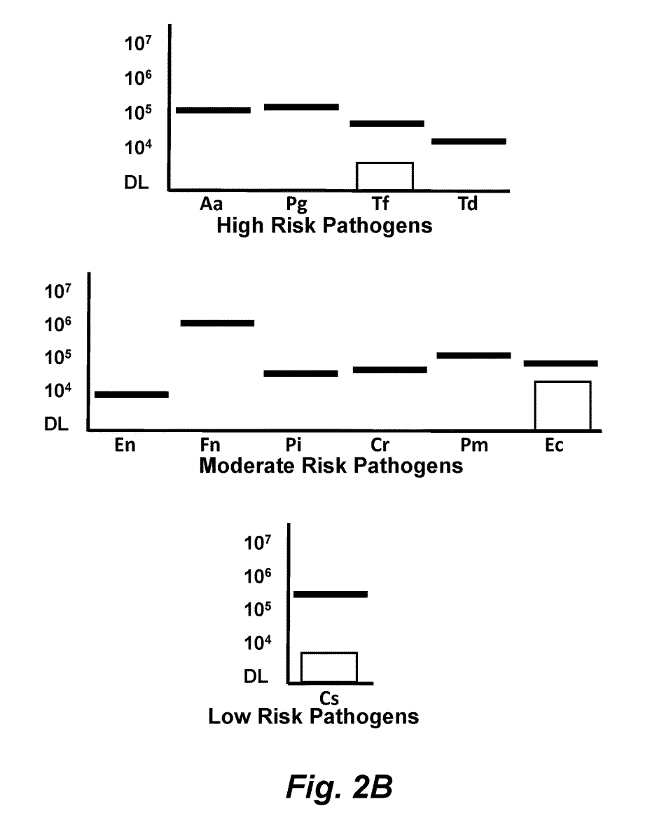

Patients were sampled before and after curettage with the composition of the disclosure, the treated site lavaged and surface samples taken for microbial analysis (Patient 1, 11 days after curretage treatment, Table 6 and FIGS. 1A and 1B; Patient 2, 13 days after curretage treatment, Table 7 and FIGS. 2A and 2B).

TABLE 6
Molecular Analysis of Periodontal and Systemic Pathogens of
Patient 1 Before and 11 Days After Treatment

| | | Level | Level | |
|---|---|---|---|---|
| Abbrev. | Species name | Before | After | Disease Association |
| Pg | Porphyromonas | High | Low | Very strong association with PD: |
| | gingivalis | | | transmittable, tissue invasive, |
| | | | | pathogenic at relatively low bacterial |
| | | | | counts. Associated with aggressive |
| | | | | forms of disease |
| Tf | Tannerella forsythia | High | Low | Very strong association with PD: |
| | | | | common pathogen associated with |
| | | | | refractory periodontitis. Strongly |
| | | | | related to increasing pocket depths. |
| Td | Treponema denticola | High | ND | Very strong association with PD: |
| | | | | invasive in cooperation with other |
| | | | | bacteria. Usually seen in combination |
| | | | | with other bacteria. |
| En | Eubacterium nodatum | High | ND | Strong association with PD: specific |
| | | | | role uncertain. Often seen in refractory |
| | | | | disease |
| Pi | Prevotella intermedia | High | Low | Strong association With PD: virulent |
| | | | | properties. Similar to Pg; often seen in |
| | | | | refractory disease |
| Fn | Fusobacterium | Low | Low | Strong association with PD: adherence |
| | nucleatum/periodonticum | | | properties to several oral pathogens; |
| | | | | often seen in refractory disease |
| Cr | Campylobacter rectus | Low | ND | Moderate association with |
| | | | | development of PD: usually found in |
| | | | | combination with other suspected |
| | | | | pathogens in refractory disease |
| Pm | Peptostreptococcus | Low | Low | Moderate association with PD: |
| | (Micromonas) micros | | | detected in higher numbers at sites of |
| | | | | active disease |
| Cs | Capnocytophaga spp | Low | High | Some association with PD: Frequently |
| | (gingavalis, ochracea | | | found in gingivitis. Often found in |
| | sputigena) | | | association with other periodontal |
| | | | | pathogens. May increase temporarily |
| | | | | following active therapy |
| Aa | Aggregatibacter | ND | ND | |
| | actinomycetemcomitans | | | |
| Ec | Eikenella corrodens | ND | ND | |

PD: Periodontal Disease
ND: Not Detected

TABLE 7
Molecular Analysis of Periodontal and Systemic Pathogens of
Patient 2 Before and 13 days After Treatment

| | | Level | Level | |
|---|---|---|---|---|
| Abbrev. | Species name | Before | After | Disease Association |
| Pg | Porphyromonas | ND | ND | Very strong association with PD^{a}: |
| | gingivalis | | | transmittable, tissue invasive, |
| | | | | pathogenic at relatively low bacterial |
| | | | | counts. Associated with aggressive |
| | | | | forms of disease |
| Tf | Tannerella forsythia | High | Low | Very strong association with PD: |
| | | | | common pathogen associated with |
| | | | | refractory periodontitis. Strongly |
| | | | | related to increasing pocket depths. |
| Td | Treponema denticola | Low | ND | Very strong association with PD: |
| | | | | invasive in cooperation with other |
| | | | | bacteria. Usually seen in combination |
| | | | | with other bacteria. |
| En | Eubacterium nodatum | High | ND | Strong association with PD: specific |
| | | | | role uncertain. Often seen in refractory |
| | | | | disease |
| Pi | Prevotella intermedia | ND | ND | Strong association With PD: virulent |
| | | | | properties. Similar to Pg; often seen in |
| | | | | refractory disease |
| Fn | Fusobacterium | Low | ND | Strong association with PD: adherence |
| | nucleatum/periodonticum | | | properties to several oral pathogens; |
| | | | | often seen in refractory disease |
| Cr | Campylobacter rectus | ND | ND | Moderate association with |
| | | | | development of PD: usually found in |
| | | | | combination with other suspected |
| | | | | pathogens in refractory disease |
| Pm | Peptostreptococcus | ND | ND | Moderate association with PD: |
| | (Micromonas) micros | | | detected in higher numbers at sites of |
| | | | | active disease |
| Cs | Capnocytophaga spp | ND | Low | Some association with PD: Frequently |
| | (gingavalis, ochracea | | | found in gingivitis. Often found in |
| | sputigena) | | | association with other periodontal |
| | | | | pathogens. May increase temporarily |
| | | | | following active therapy |
| Aa | Aggregatibacter | ND | ND | |
| | actinomycetemcomitans | | | |
| Ec | Eikenella corrodens | ND | ND | |

PD: Periodontal Disease
ND: Not Detected
Note significant reduction of pathogenic bacteria after using the composition of the disclosure as an adjunct to scaling and root planing and debridment of periodontal defects.

1. A periodontal adjunct composition consisting of potassium hydroxide, a metal salt, a phenolic compound, an H₂ receptor antagonist, and an essential oil, wherein the potassium hydroxide is between about 54% and about 95% by weight; the metal salt is between about 0.5% and about 5% by weight; the phenolic compound is between about 0.5% and about 5% by weight; the essential oil is between about 0.5% and about 5% by weight; and the H₂ receptor antagonist is between about 0.1% and about 1% by weight, wherein % by weight is % by weight of the final composition weight.

2. The periodontal adjunct composition of claim 1, wherein the composition is admixed with an excipient.

3. (canceled)

4. The periodontal adjunct composition of claim 1, wherein the H₂ receptor antagonist is selected from the group consisting of: cimetidine, nizatidine, famotidine, and ranitidine.

5. (canceled)

6. The periodontal adjunct composition of claim 1, wherein the phenolic compound is selected from the group consisting of thymol, carvacrol, cresol, estradiol, eugenol, gallic acid, guaiacol, methyl salicylate, raspberry ketone, salicylic acid, sesamol, tumeric, phenol, bisphenol A, BHT, 4-nonylphenol, orthophenyl phenol, phenolphthalein, and xylenol.

7. The periodontal adjunct composition of claim 1, wherein the essential oil is selected from the group consisting of cinnamon oil, clove oil, eucalyptus oil, peppermint oil, oregano oil, lavender oil, tea tree oil, bergamont oil, lemongrass oil, thyme oil, basil oil, and rosemary oil.

8. The periodontal adjunct composition of claim 2, wherein the excipient is glycerin.

9. The periodontal adjunct composition of claim 2, wherein the periodontal adjunct composition is a paste.

10. The periodontal adjunct composition of claim 1, wherein:
the potassium hydroxide is between about 85% and about 95% by weight;
the metal salt is between about 1% and about 5% by weight;
the phenolic compound is between about 1% and about 5% by weight; and
the essential oil is between about 1% and about 5% by weight, wherein % by weight is % weight of the final composition weight.

11-12. (canceled)

13. The periodontal adjunct composition of claim 10, wherein the potassium hydroxide is about 90% by weight; the metal salt is about 3% by weight; the phenolic compound is about 3% by weight; and the essential oil is about 3% by weight, wherein % by weight is % weight of the final composition weight.

14. The periodontal adjunct composition of claim 2, wherein the potassium hydroxide is about 60% by weight; the metal salt is about 2% by weight; the phenolic compound is about 2% by weight; the essential oil is about 2% by weight; and the composition further comprises about 40% by weight of the excipient, wherein % by weight is % weight of the final composition weight.

15. The periodontal adjunct composition of claim 1, wherein the metal salt is magnesium carbonate, the phenolic compound is thymol, and the essential oil is cinnamon oil, clove oil, or peppermint oil.

16. The periodontal adjunct composition of claim 1, wherein the phenolic compound is thymol, and the essential oil is one of cinnamon oil, clove oil, and peppermint oil.

17. The periodontal adjunct composition of claim 8, wherein the periodontal adjunct composition consists of about 60% by weight of potassium hydroxide, about 2% by weight of magnesium carbonate, about 2% by weight of thymol, about 2% by weight of one of cinnamon oil, clove oil, or peppermint oil, about 0.33% by weight of an H₂ receptor antagonist, and about 33% by weight of glycerin, wherein % by weight is % weight of the final composition weight.

18. A method of reducing an oral microbial population at a site of periodontal treatment, comprising the steps of
(a) applying to the surface of a site of periodontal treatment of a patient an amount of a periodontal adjunct composition consisting of:
between about 50% and about 70% by weight of at least one alkali metal hydroxide;
between about 0.5% and about 5% by weight of a metal salt;
between about 0.5% and about 5% by weight of a phenolic compound;
between about 0.5% and about 5% by weight of an essential oil;
between about 5% and about 40% by weight of an excipient; and
between about 0.1% and about 1% by weight of an H₂ receptor antagonist,
wherein the periodontal adjunct composition is a paste, and wherein % by weight is % weight of the final composition weight; and
(b) rinsing the periodontal adjunct composition from the site of application with a physiologically acceptable liquid.

19. (canceled)

20. The method of claim 18, further comprising the step of obtaining the periodontal adjunct composition by admixing the at least one alkali metal hydroxide, the metal salt, the phenolic compound, the essential oil, and the excipient to form a paste.

21. The method of claim 18, wherein:
the at least one alkali metal hydroxide is selected from the group consisting of potassium hydroxide, sodium hydroxide, calcium hydroxide, lithium hydroxide, and rubidium hydroxide;
the phenolic compound is selected from the group consisting of thymol, carvacrol, cresol, estradiol, eugenol, gallic acid, guaiacol, methyl salicylate, raspberry ketone, salicylic acid, sesamol, tumeric, phenol, bisphenol A, BHT, 4-nonylphenol, orthophenyl phenol, phenolphthalein, and xylenol; and
the essential oil is selected from the group consisting of cinnamon oil, clove oil, eucalyptus oil, peppermint oil, oregano oil, lavender oil, tea tree oil, bergamont oil, lemongrass oil, thyme oil, basil oil, and rosemary oil.

22. The method of claim 18, wherein the at least one alkali metal hydroxide is potassium hydroxide, the metal salt is magnesium carbonate the phenolic compound is thymol, the essential oil is one of cinnamon oil, clove oil, or peppermint oil, and the excipient is glycerin.

23. The method of claim 22, wherein the periodontal adjunct composition consists of about 60% by weight of potassium hydroxide, about 2% by weight of magnesium carbonate, about 2% by weight of thymol, about 2% by weight of cinnamon oil, clove oil, or peppermint oil, and about 33% by weight of glycerin, wherein % by weight is % weight of the final composition weight.

24. The method of claim 18, wherein the periodontal adjunct composition consists of about 60% by weight of potassium hydroxide, about 2% by weight of magnesium carbonate, about 2% by weight of thymol, about 2% by weight of cinnamon oil, clove oil, or peppermint oil, about 0.33% of an H₂ receptor antagonist, and about 40% by weight of glycerin, wherein % by weight is % weight of the final composition weight.
